(19) United States
(12) Patent Application Publication
(10) Pub. No.: US 2008/0114408 A1
(43) Pub. Date: May 15, 2008

(54) METHOD AND DEVICE FOR SIMULATED EXERCISE

(76) Inventors: Allan C. Shuros, St. Paul, MN (US); Joseph M. Pastore, Woodbury, MN (US); Jeffrey A. Von Arx, Minneapolis, MN (US); Julio C. Spinelli, Shoreview, MN (US); William J. Linder, Golden Valley, MN (US)

Correspondence Address: SCHWEGMAN, LUNDBERG & WOESSNER, P.A., P.O. BOX 2938, MINNEAPOLIS, MN, 55402, US

(21) Appl. No.: 11/559,131

(22) Filed: Nov. 13, 2006

(51) Int. Cl. A61N 1/37

(52) U.S. Cl. 607/11

(57) ABSTRACT

A device and method for delivering pacing therapy to the heart in order to improve cardiac function in heart failure and post-MI patients. The pacing therapy is delivered in a manner that mimics the effects of exercise and improves symptoms even in patients who are exercise intolerant. The simulated exercise pacing may be delivered on an intermittent basis in accordance with a defined schedule and/or in response to detected conditions or events.

### FIELD OF THE INVENTION

This invention pertains to apparatus and methods for the treatment of heart disease and to devices providing electrostimulation to the heart such as cardiac pacemakers.

### BACKGROUND

Heart failure (HF) is a debilitating disease that refers to a clinical syndrome in which an abnormality of cardiac function causes a below normal cardiac output that can fall below a level adequate to meet the metabolic demand of peripheral tissues. Heart failure can be due to a variety of etiologies with ischemic heart disease being the most common. Inadequate pumping of blood into the arterial system by the heart is sometimes referred to as “forward failure,” with “backward failure” referring to the resulting elevated pressures in the lungs and systemic veins which lead to congestion. Backward failure is the natural consequence of forward failure as blood in the pulmonary and venous systems fails to be pumped out. Forward failure can be caused by impaired contractility of the ventricles due, for example, to coronary artery disease, or by an increased afterload (i.e., the forces resisting ejection of blood) due to, for example, systemic hypertension or valvular dysfunction. One physiological compensatory mechanism that acts to increase cardiac output is due to backward failure which increases the diastolic filling pressure of the ventricles and thereby increases the preload (i.e., the degree to which the ventricles are stretched by the volume of blood in the ventricles at the end of diastole). An increase in preload causes an increase in stroke volume during systole, a phenomena known as the Frank-Starling principle. Thus, heart failure can be at least partially compensated by this mechanism but at the expense of possible pulmonary and/or systemic congestion.

When the ventricles are stretched due to the increased preload over a period of time, the ventricles become dilated. The enlargement of the ventricular volume causes increased ventricular wall stress at a given systolic pressure. Along with the increased pressure-volume work done by the ventricle, this acts as a stimulus for hypertrophy of the ventricular myocardium which leads to alterations in cellular structure, a process referred to as ventricular remodeling. Ventricular remodeling leads to further dysfunction by decreasing the compliance of the ventricles (thereby increasing diastolic filling pressure to result in even more congestion) and causing eventual wall thinning that causes further deterioration in cardiac function. It has been shown that the extent of ventricular remodeling is positively correlated with increased mortality in HF patients.

A myocardial infarction (MI) is the irreversible damage done to a segment of heart muscle by ischemia, where the myocardium is deprived of adequate oxygen and metabolite removal due to an interruption in blood supply. It is usually due to a sudden thrombotic occlusion of a coronary artery, commonly called a heart attack. If the coronary artery becomes completely occluded and there is poor collateral blood flow to the affected area, a transmural or full-wall thickness infarct can result in which much of the contractile function of the area is lost. Over a period of one to two months, the necrotic tissue heals, leaving a scar. The most extreme example of this is a ventricular aneurysm, where all of the muscle fibers in the area are destroyed and replaced by fibrous scar tissue. Even if the ventricular dysfunction as a result of the infarct is not immediately life-threatening, a common sequela of a transmural myocardial infarction, or any major MI, especially in the left ventricle, is heart failure brought about by ventricular remodeling in response to the hemodynamic effects of the infarct that causes changes in the shape and size of the ventricle. The remodeling is initiated in response to a redistribution of cardiac stress and strain caused by the impairment of contractile function in the infarcted area as well as in nearby and/or interspersed viable myocardial tissue with lessened contractility due to the infarct. Following an MI, the infarcted area includes tissue undergoing ischemic necrosis and is surrounded by normal myocardium. Until scar tissue forms and even after it forms, the area around the infarcted area is particularly vulnerable to the distending forces within the ventricle and undergoes expansion over a period of hours to days. Over the next few days and months after scar tissue has formed, global remodeling and chamber enlargement occur due to complex alterations in the architecture of the ventricle involving both infarcted and non-infarcted areas. It has been found that the extent of left ventricular remodeling in the late period after an infarction, as represented by measurements of end-systolic and end-diastolic left ventricular volumes, is an even more powerful predictor of subsequent mortality than the extent of coronary artery disease.

Remodeling is thought to be the result of a complex interplay of hemodynamic, neural, and hormonal factors that occur primarily in response to myocardial wall stress. As noted above, one physiological compensatory mechanism that acts to increase cardiac output is increased diastolic filling pressure of the ventricles as an increased volume of blood is left in the lungs and venous system, thus increasing preload. The ventricular dilation resulting from the increased preload causes increased ventricular wall stress at a given systolic pressure in accordance with Laplace's law. Along with the increased pressure-volume work done by the ventricle, this acts as a stimulus for compensatory hypertrophy of the ventricular myocardium. Hypertrophy can increase systolic pressures but, if the hypertrophy is not sufficient to meet the increased wall stress, further and progressive dilation results. This non-compensatory dilation causes wall thinning and further impairment in left ventricular function. It also has been shown that the sustained stresses causing hypertrophy may induce apoptosis (i.e., programmed cell death) of cardiac muscle cells. Thus, although ventricular dilation and hypertrophy may at first be compensatory and increase cardiac output, the process ultimately results in further deterioration and dysfunction.

It has long been known that the heart muscle responds favorably to exercise so as to result in greater pumping efficacy. Studies have shown that HF and post-MI patients can improve their cardiac function and prognosis with regular periods of exercise. Many HF and post-MI patients, however, are either debilitated and cannot exercise or do not tolerate exercise well enough to exercise effectively.

### BRIEF DESCRIPTION OF THE DRAWINGS

FIG. 1 illustrates the physical configuration of an exemplary pacing device.

FIG. 2 shows the components of an exemplary device.

FIG. 3 is a block diagram of the electronic circuitry of an exemplary device.

FIG. 4 illustrates an exemplary algorithm for implementing intermittent simulated exercise pacing.

FIGS. 5 through 8 illustrate an embodiment of a pacing device integrated into a lead.

### DETAILED DESCRIPTION

Clinical studies have shown that HF and post-MI patients who follow a regular (e.g. 20 min/day, 3 times a week) exercise regimen have symptomatic improvement compared to those who are sedentary. However, not all HF and post-MI patients can exercise due to their cardiac disease or other debilitating conditions. This disclosure describes methods and devices that use short durations of pacing therapy designed to mimic exercise in order to provide protection from heart failure development and/or attenuation/reversal of cardiac disease progression.

When cardiac output is insufficient to meet the increased metabolic demand, the body responds to the situation with increased activity of the sympathetic nervous system that, among other things, increases heart rate, myocardial contractility, and blood volume. Although acutely beneficial, the long-term effects of increased sympathetic activity are deleterious and lead to ventricular remodeling such as described above. A characteristic feature of chronic cardiac disease is an abnormal autonomic tone with an attenuated level of parasympathetic activity relative to sympathetic activity. When the heart is stressed on a periodic short-term basis, however, such as occurs with regular exercise, the effect is beneficial on both myocardial function and autonomic tone, leading to an increased level of parasympathetic activity. In order to mimic the effects of exercise, pacing therapy can be delivered on a short-term basis in a manner that stresses the heart similar to exercise. Such pacing therapy is referred to herein as simulated exercise pacing. Simulated exercise pacing may generally involve pacing the heart in a manner that temporarily compromises cardiac output by producing relatively inefficient ventricular contractions and/or some degree of atrio-ventricular dyssynchrony.

As described below, a device for delivering such simulated exercise pacing may be a device with the capability of also delivering bradycardia pacing, CRT, cardioversion/defibrillation shocks, and/or neural stimulation (e.g., vagal nerve stimulation). The device may be programmed to deliver simulated exercise pacing for a prescribed amount of time per day (e.g. 30 min). The time when therapy delivery is started may be random (once per day at a random time), at a specific time each day, or triggered by a specific event (e.g. when the patient falls asleep, the patient wakes up, or the patient's exertion level falls below a certain threshold).

### 1. Exemplary Cardiac Device

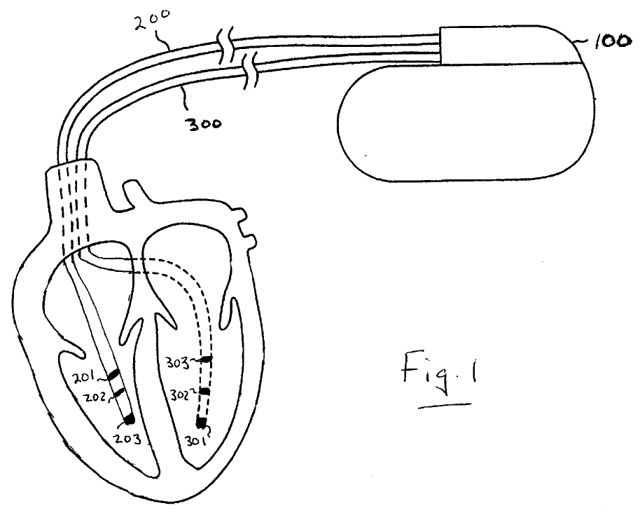

FIG. 1 shows an implantable cardiac device 100 for delivering simulated exercise therapy as well as possibly other types of pacing therapy. Implantable pacing devices are typically placed subcutaneously or submuscularly in a patient's chest with leads threaded intravenously into the heart to connect the device to electrodes disposed within a heart chamber that are used for sensing and/or pacing of the chamber. Electrodes may also be positioned on the epicardium by various means. A programmable electronic controller causes the pacing pulses to be output in response to lapsed time intervals and/or sensed electrical activity (i.e., intrinsic heart beats not as a result of a pacing pulse). The device senses intrinsic cardiac electrical activity through one or more sensing channels, each of which incorporates one or more of the electrodes. In order to excite myocardial tissue in the absence of an intrinsic beat, pacing pulses with energy above a certain threshold are delivered to one or more pacing sites through one or more pacing channels, each of which incorporates one or more of the electrodes. FIG. 1 shows the exemplary device having two leads 200 and 300, each of which is a multi-polar (i.e., multi-electrode) lead having electrodes 201-203 and 301-303, respectively. The electrodes 201-203 are disposed in the right ventricle in order to excite or sense right ventricular or septal regions, while the electrodes 301-303 are disposed in the coronary sinus in order to excite or sense regions of the left ventricle. Other embodiments may use any number of electrodes in the form of unipolar and/or multi-polar leads in order to excite different myocardial sites. As explained below, once the device and leads are implanted, the pacing and/or sensing channels of the device may be configured with selected ones of the multiple electrodes in order to selectively pace or sense a particular myocardial site(s).

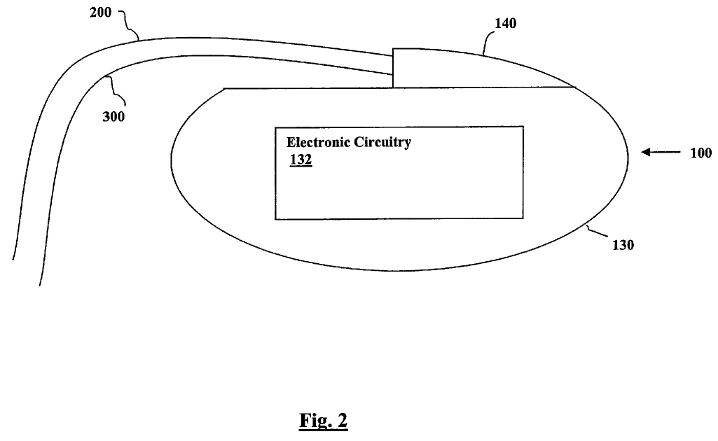

FIG. 2 shows the components of the implantable device 100 in more detail. The implantable device 100 includes a hermetically sealed housing 130 that is placed subcutaneously or submuscularly in a patient's chest. The housing 130 may be formed from a conductive metal, such as titanium, and may serve as an electrode for delivering electrical stimulation or sensing in a unipolar configuration. A header 140, which may be formed of an insulating material, is mounted on the housing 130 for receiving leads 200 and 300 which may be then electrically connected to pulse generation circuitry and/or sensing circuitry. Contained within the housing 130 is the electronic circuitry 132 for providing the functionality to the device as described herein which may include a power supply, sensing circuitry, pulse generation circuitry, a programmable electronic controller for controlling the operation of the device, and a telemetry transceiver capable of communicating with an external programmer or a remote monitoring device.

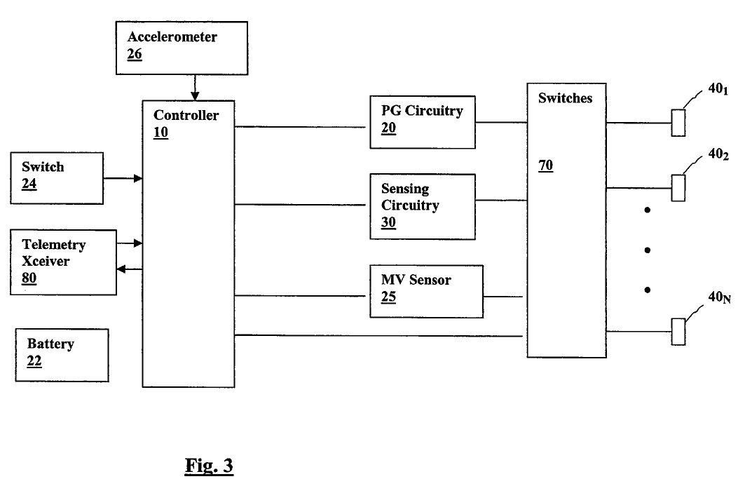

FIG. 3 shows a system diagram of the electronic circuitry 132. A battery 22 supplies power to the circuitry. The controller 10 controls the overall operation of the device in accordance with programmed instructions and/or circuit configurations. The controller may be implemented as a microprocessor-based controller and include a microprocessor and memory for data and program storage, implemented with dedicated hardware components such as ASICs (e.g., finite state machines), or implemented as a combination thereof. The controller also includes timing circuitry such as external clocks for implementing timers used to measure lapsed intervals and schedule events. As the term is used herein, the programming of the controller refers to either code executed by a microprocessor or to specific configurations of hardware components for performing particular functions. Interfaced to the controller are sensing circuitry 20 and pulse generation circuitry 30 by which the controller interprets sensing signals and controls the delivery of paces in accordance with a pacing mode. The controller also implements timers derived from external clock signals in order to keep track of time and implement real-time operations such as scheduled simulated exercise pacing.

The sensing circuitry 20 receives atrial and/or ventricular electrogram signals from sensing electrodes and includes sensing amplifiers, analog-to-digital converters for digitizing sensing signal inputs from the sensing amplifiers, and registers that can be written to for adjusting the gain and threshold values of the sensing amplifiers. The sensing circuitry of the pacemaker detects a chamber sense, either an atrial sense or ventricular sense, when an electrogram signal (i.e., a voltage sensed by an electrode representing cardiac electrical activity) generated by a particular channel exceeds a specified detection threshold. Pacing algorithms used in particular pacing modes employ such senses to trigger or inhibit pacing, and the intrinsic atrial and/or ventricular rates can be detected by measuring the time intervals between atrial and ventricular senses, respectively.

The pulse generation circuitry 30 delivers pacing pulses to pacing electrodes disposed in the heart and includes capacitive discharge or current source pulse generators, registers for controlling the pulse generators, and registers for adjusting pacing parameters such as pulse energy (e.g., pulse amplitude and width). The device allows adjustment of the pacing pulse energy in order to ensure capture of myocardial tissue (i.e., initiating of a propagating action potential) by a pacing pulse. The pulse generation circuitry may also include a shocking pulse generator for delivering a defibrillation/cardioversion shock via a shock electrode upon detection of a tachyarrhythmia.

A telemetry transceiver 80 is interfaced to the controller which enables the controller to communicate with an external device such as an external programmer and/or a remote monitoring unit. An external programmer is a computerized device with an associated display and input means that can interrogate the pacemaker and receive stored data as well as directly adjust the operating parameters of the pacemaker. The external device may also be a remote monitoring unit that may be interfaced to a patient management network enabling the implantable device to transmit data and alarm messages to clinical personnel over the network as well as be programmed remotely. The network connection between the external device and the patient management network may be implemented by, for example, an internet connection, over a phone line, or via a cellular wireless link. A magnetically or tactilely actuated switch 24 is also shown as interfaced to the controller in this embodiment to allow the patient to signal certain conditions or events to the implantable device. The controller may be programmed to use actuation of the switch 24 to initiate and/or cease exercise simulation pacing.

A pacing channel is made up of a pulse generator connected to an electrode, while a sensing channel is made up of a sense amplifier connected to an electrode. Shown in the figure are electrodes 40₁ through 40_{N} where N is some integer. The electrodes may be on the same or different leads and are electrically connected to a MOS switch matrix 70. The switch matrix 70 is controlled by the controller and is used to switch selected electrodes to the input of a sense amplifier or to the output of a pulse generator in order to configure a sensing or pacing channel, respectively. The device may be equipped with any number of pulse generators, amplifiers, and electrodes that may be combined arbitrarily to form sensing or pacing channels. The device is therefore capable of delivering single-site or multiple site ventricular pacing for purposes of exercise simulation as well as conventional pacing. One or more pacing channels may also be configured, by appropriate lead placement and pulse energy/frequency settings, for delivering electrical stimulation to stimulate sympathetic and/or parasympathetic nerves. For example, a lead with a stimulation electrode may be placed in proximity to the vagus nerve in order to stimulate that nerve and increase parasympathetic activity. The switch matrix 70 also allows selected ones of the available implanted electrodes to be incorporated into sensing and/or pacing channels in either unipolar or bipolar configurations. A bipolar sensing or pacing configuration refers to the sensing of a potential or output of a pacing pulse between two closely spaced electrodes, where the two electrodes are usually on the same lead (e.g., a ring and tip electrode of a bipolar lead or two selected electrodes of a multi-polar lead). A unipolar sensing or pacing configuration is where the potential sensed or the pacing pulse output by an electrode is referenced to the conductive device housing or another distant electrode.

The controller is capable of operating the device in a number of programmed pacing modes which define how pulses are output in response to sensed events and expiration of time intervals. Most pacemakers for treating bradycardia are programmed to operate synchronously in a so-called demand mode where sensed cardiac events occurring within a defined interval either trigger or inhibit a pacing pulse. Inhibited demand pacing modes utilize escape intervals to control pacing in accordance with sensed intrinsic activity such that a pacing pulse is delivered to a heart chamber during a cardiac cycle only after expiration of a defined escape interval during which no intrinsic beat by the chamber is detected. Escape intervals for ventricular pacing can be restarted by ventricular or atrial events, the latter allowing the pacing to track intrinsic atrial beats and/or follow atrial paces. An exertion level sensor (such as the accelerometer 26 or the minute ventilation sensor 25 shown in FIG. 3 or other sensor that measures a parameter related to metabolic demand) enables the controller to adapt the pacing rate in accordance with changes in the patient's physical activity. As described below, the exertion level sensor may also be used in scheduling delivery of simulated exercise pacing.

The pacing device just described may be configured to deliver pacing therapy in a number of pacing modes with different configurations of pacing channels and different pacing parameter settings. For example, the device may be programmed to deliver single-site ventricular pacing, biventricular pacing, or multi-site ventricular pacing. Such pacing may be delivered using atrial tracking modes (e.g., DDD or VDD) and AV sequential modes (e.g., DVI, DDI) where a ventricular pace(s) is delivered upon expiration of an AV delay interval following an atrial sense or pace if no ventricular sense occurs before expiration. Pacing can also be delivered in non-atrial tracking modes such as VVI where a pace is delivered upon expiration of a ventricular escape interval started by a ventricular sense or pace if no ventricular sense occurs before expiration. These pacing modes are examples of bradycardia pacing modes because they were originally developed to treat bradycardia by enforcing some minimum heart rate. Pacing delivered in conjunction with such bradycardia pacing modes, however, can also be used to treat conditions other than bradycardia. It has been shown that some heart failure patients suffer from intraventricular and/or interventricular conduction defects (e.g., bundle branch blocks) such that their cardiac outputs can be increased by improving the synchronization of ventricular contractions with electrical stimulation. In order to treat these problems, implantable cardiac devices have been developed that provide appropriately timed electrical stimulation to one or more heart chambers in an attempt to improve the coordination of atrial and/or ventricular contractions, termed cardiac resynchronization therapy (CRT). Ventricular resynchronization is useful in treating heart failure because, although not directly inotropic, resynchronization can result in a more coordinated contraction of the ventricles with improved pumping efficiency and increased cardiac output. Currently, a most common form of CRT applies stimulation pulses to both ventricles, either simultaneously or separated by a specified biventricular offset interval, and after a specified atrio-ventricular delay interval with respect to the detection of an intrinsic atrial contraction or delivery of an atrial pace.

### 2. Delivery of Simulated Exercise Pacing

As discussed above, myocardial infarction and/or heart failure can cause deleterious ventricular remodeling. It has been shown that remodeling and/or patient quality of life can be improved in MI/HF patients with a regimen of regular exercise (e.g., 30 minutes a day for 3 times a week). Thus, short intervals of stress to the body provide a chronic benefit (i.e., a training effect). A similar benefit may be elicited by applying short intervals of stress isolated to the ventricles with intermittent pacing therapy designed to compromise cardiac output, referred to herein as simulated exercise pacing. Simulated exercise pacing may include any or all of the following: 1) pacing a ventricle in an atrial tracking or AV sequential pacing mode using a shortened AV delay that causes diminished left ventricular filling during diastole, 2) pacing a ventricle in a VVI mode to limit the atrial contraction contribution to left ventricular filling during diastole, 3) pacing one or both ventricles at a site or sites that produce an asynchronous and inefficient ventricular contraction, 4) biventricular pacing with an interventricular pacing delay designed to produce inefficient cardiac contractions, and 5) rapid pacing (atrial or ventricular) using any pacing mode at a rate that prevents adequate ventricular filling. In response to such simulated exercise pacing, the body compensates for the reduced cardiac output by increasing heart rate, increasing myocardial contractility, and/or increasing blood volume. Stressing the heart continuously in this manner would be detrimental, but delivering additional stress intermittently provides a training effect and is beneficial to the heart. Simulated exercise pacing may be applied, for example, for a few minutes each day or delivered intermittently on some other basis. Following these short periods of stress, the parasympathetic system may be activated, thereby providing therapeutic benefit. Similar to the benefits that transient exercise provides the entire body, intermittent stress localized to the ventricles brought about by simulated exercise pacing allows the heart to become stronger and more resistant to future stressful situations.

Stressing the heart by delivering simulated exercise pacing as described above may or may not cause a drop in cardiac output, depending upon how an individual patient responds. Some patients, for example, may be capable of responding to the stress of simulated exercise pacing in a manner that prevents a significant decrease in cardiac output. In other patients, after an MI or during heart failure, the compromised ventricles may be less then capable of maintaining normal cardiac output. A feedback mechanism may be implemented that analyze cardiac performance indicators and modulates delivery of the therapy accordingly. For example, a cardiac output sensor may be incorporated into the device that measures ventricular volumes using an impedance technique. Simulated exercise pacing may then be delivered or not delivered in accordance with whether the cardiac output is found to be above or below a specified threshold value, where the specified threshold may be made to be dependent upon a measured exertion level. As described below, simulated exercise pacing may also be initiated and/or ceased depending upon the detection of certain conditions related to the patient's physiological status. Simulated exercise pacing therapy may also be combined with other device therapies such as bradycardia, tachycardia, cardiac resynchronization, or post-MI pacing.

As described above, pacing can be delivered to the heart in a way that mimics the beneficial effects of exercise. Chronic simulated exercise pacing, however, would overstress the heart in HF or post-MI patients and could be hazardous. Accordingly, simulated exercise pacing should be delivered on an intermittent basis. A device such as shown in FIGS. 1 and 2 can be configured to deliver simulated exercise pacing by switching from a normal operating mode to a simulated exercise mode according to some defined schedule that specifies switching in response to lapsed time intervals and/or in response to one or more particular triggering events or conditions. If the device is configured to switch to the simulated exercise mode in response to a triggering event or condition, some limit could be imposed on the amount of stimulation delivered over a specified period of time. In the normal operating mode, the device may deliver no therapy at all or may be configured to delivery therapies such as bradycardia pacing or cardiac resynchronization pacing. After switching to the simulated exercise mode, the device may then deliver the pacing using one or more pacing modes, configurations, and/or parameter settings different from pacing delivered during the normal operating mode. The simulated exercise mode may also allow therapies of the normal operating mode to continue such as bradycardia pacing, cardiac resynchronization pacing, and/or shocks or anti-tachycardia pacing in response to detection of tachyarrhythmias.

In order to provide intermittent simulated exercise pacing, the device switches from its normal operating mode to the simulated exercise mode based upon lapsed time intervals and/or in response to detection of one or more particular triggering conditions or events. In another embodiment, the device may switch to the simulated exercise mode upon receiving a command to do so for some specified period of time, where such a command may be received from an external programmer, or received via a patient management network. A defined schedule may specify switching to the simulated exercise mode at periodic intervals (e.g., for five minutes each day) or at a random time during each day or other specified time period. Such a defined schedule could also specify a time for switching to the simulated exercise mode when a patient is expected to be awake or when a patient is expected to be sleeping. A defined schedule may also prescribe an amount of time over a specified time period for which the device is to operate in the simulated exercise mode. For example, the defined schedule may prescribe that simulated exercise pacing be delivered for one hour each day. The controller may then be programmed to opportunistically switch to the simulated exercise mode when one or more specified triggering conditions are met in order to meet the prescriptions of the defined schedule. Examples of possible triggering conditions are a measured exertion level being within a specified entry range, a measured heart rate being within a specified entry range, and actuation of a magnetically or tactilely actuated switch incorporated into the device by the patient that initiates simulated exercise pacing. In such embodiments, the simulated exercise pacing delivered in response to the triggering events may then be limited in amount or duration over some specified period of time. For example, the device could be programmed to deliver no more than 30 minutes of simulated exercise pacing per day in response to such triggering events.

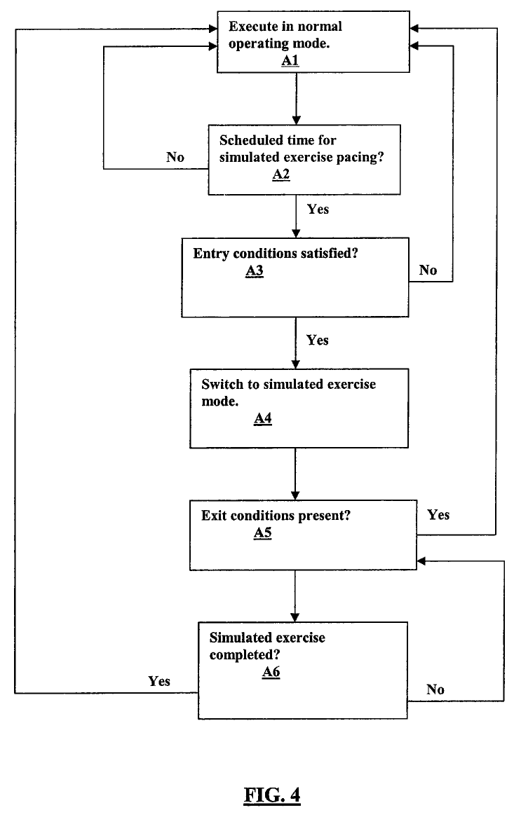

FIG. 4 illustrates one way that simulated exercise pacing may be implemented by a cardiac device. In this embodiment, the controller of the device is programmed to transition through a number of different states, designated as A1 through A6. At state A1, the device operates in its normal operating mode. At state A2, while continuing to operate in state A1, the device determines whether it should switch to the simulated exercise mode based upon a lapsed time interval or a triggering condition. Optionally, the device may also be configured to test for one or more particular entry conditions before switching to the simulated exercise mode as implemented by state A3. Examples of entry conditions that must be satisfied before the switch to the simulated exercise mode include a measured exertion level being within a specified entry range, a measured heart rate being within a specified entry range, non-detection of cardiac arrhythmias, non-detection of cardiac ischemia, and actuation of a magnetically or tactilely actuated switch incorporated into the device by the patient that allows delivery of simulated exercise pacing. At state A3, the device checks to see if the one or more entry conditions are satisfied and returns to state A1 if not. If the appropriate entry conditions are satisfied, the device switches to the simulated exercise mode at state A4. As discussed above, the simulated exercise mode supercedes the normal operating mode to the extent necessary to carry out the simulated exercise pacing but may allow certain functions performed in the normal operating mode to continue. Alternatively, the simulated exercise mode could be said to incorporate particular functions of the normal operating mode, which functions are modified if necessary to deliver the simulated exercise pacing. While executing in the simulated exercise mode, the device may optionally be configured to monitor for one or more exit conditions which cause the device to revert to the normal operating mode. Such exit conditions could be the same or different from the entry conditions that must be satisfied before entering the simulated exercise mode. At state A5, while executing in the simulated exercise mode, the device monitors for the occurrence of one or more exit conditions such as a measured exertion level being outside a specified permissible range, a measured heart rate being outside a specified permissible range, presence of a cardiac arrhythmia, presence of cardiac ischemia, and actuation of a magnetically or tactilely actuated switch incorporated into the device by the patient to stop delivery of simulated exercise pacing. If an exit condition occurs, the device returns to the normal operating mode at state A1. Otherwise, the device proceeds to state A6 and checks to see if the prescribed amount and/or duration of simulated exercise pacing has been delivered. If the specified amount or duration of simulated exercise pacing has been delivered, the device returns to state A1 and resumes the normal operating mode. Otherwise, the device loops back to state A5 to monitor for exit conditions.

In order to reliably provide the desired hemodynamic effects when switched to the simulated exercise mode, the device can be programmed use escape intervals designed for that purpose during the simulated exercise mode. For example, the simulated exercise pacing may be delivered to the ventricles in an atrial triggered synchronous mode (e.g., DDD or VDD) with predefined atrio-ventricular (AV) and ventricular-ventricular (VV) escape intervals or in a non-atrial triggered ventricular pacing mode (e.g., VVI) with a pre-defined ventricular escape interval where the length of the escape intervals may be set to values which result in a diminished end-diastolic ventricular filling volume. It may be desirable, however, to incorporate additional steps into the algorithm before switching. For example, the escape intervals for the simulated exercise mode may be dynamically determined before the mode switch in order to ensure the desired hemodynamic effect. In an embodiment where the simulated exercise mode is a non-atrial triggered pacing mode, the device may measure the patient's intrinsic heart rate before the mode switch and then set the ventricular escape interval so that the pacing rate for the simulated exercise pacing mode is higher than the intrinsic rate. If the patient is receiving rate-adaptive ventricular pacing therapy in the normal operating mode, the ventricular escape interval for the simulated exercise pacing mode may be similarly modulated by an exertion level measurement. In an embodiment where the simulated exercise pacing is delivered in an atrial triggered pacing mode, the device may measure the patient's intrinsic AV interval before the mode switch (e.g., as an average over a number of cycles preceding the mode switch) so that the AV escape interval for delivering ventricular pacing can be set to a value that results in some degree of atrio-ventricular dyssynchrony.

### 3. Detection of Triggering, Entry, and Exit Conditions

As discussed above, it may be desirable for the device to switch to the simulated exercise mode according to a defined schedule only if one or more specified entry conditions are satisfied. Whether or not entry conditions are employed, it may also be desirable for the device to exit the simulated exercise mode if one or more specified exit conditions occur. Finally, a defined schedule for switching to the simulated exercise mode may employ one or more specified triggering conditions that when satisfied cause the mode switch. Discussed below are examples of conditions that can be detected by appropriately configured implantable device and used as entry, exit, and/or triggering conditions.

One example of a triggering and/or entry condition is if the measured exertion level is within a specified range, where the exertion level may be measured, for example, as minute ventilation with a minute ventilation sensor, as an activity level with an accelerometer, or some combination of such measurements. Another example of a triggering and/or entry condition is if the patient's heart rate is within a specified range, where the heart rate is measured via a cardiac sensing channel. With some patients, it may be desirable for the simulated exercise mode to take place when the patient is not active as reflected by a measured exertion level and/or heart rate below a specified value. With other patients, on the other hand, it may be desirable to switch to the simulated exercise mode only when the patient is deemed to be active, as determined by an exertion level and/or heart rate above a specified value. A measured exertion level and/or heart rate either above or below a specified value may also be used as a triggering event to initiate the simulated exercise mode for some specified period of time. A measured exertion level or heart rate may also be used as an exit condition such that the device is programmed to revert from the simulated exercise mode back to the normal operating mode if the measured exertion level and/or heart rate falls outside of a specified permissible range.

It may also be desirable to inhibit a switch to the simulated exercise mode and/or revert to the normal operating mode if the patient is presently experiencing some degree of cardiac ischemia and/or a cardiac arrhythmia is detected. The device may be configured to detect cardiac ischemia from a morphology analysis of an electrogram collected during an intrinsic or a paced beat, the latter sometimes referred to as an evoked response. The electrogram for detection of ischemia is recorded from a sensing channel that senses the depolarization and repolarization of the myocardium during a cardiac cycle. The sensing channel used for this purpose may be a sensing channel used for detecting cardiac arrhythmias and/or intrinsic beats or may be a dedicated channel. In order to detect an ischemic change, the electrogram can be compared with a reference electrogram to see if a current of injury is present. The comparison may involve, for example, cross-correlating the recorded and reference electrograms or comparing ST segment amplitudes, slopes, or integrations with reference values. If a change in a recorded electrogram indicative of ischemia is detected and/or a cardiac arrhythmia is detected, the controller may be programmed to inhibit switching to simulated exercise mode and/or programmed to revert back to the normal operating mode. Detection of cardiac ischemia or cardiac arrhythmias may also be logged as clinically significant events in the pacemaker's memory, where the event log and/or the recorded electrogram exhibiting the ischemia or arrhythmia may then be later downloaded to a clinician for analysis via an external programmer and/or a patient management network. Information derived from other analyses or other sensing modalities may also be used to more specifically detect cardiac ischemia. For example, dyspnea or other abnormal breathing patterns may be detected using a minute ventilation sensor by programming the controller to compare the transthoracic impedance signal from the sensor with a template representing the abnormal pattern.

### 4. Integrated Lead Embodiment

In many instances, simulated exercise pacing as described above may be only needed for some temporary period of time. For example, some post-MI and HF patients may over time recover sufficiently so that they can exercise normally and do not need simulated exercise. For these patients, a conventionally implanted pacing device such as illustrated in FIGS. 1 and 2 may be implanted and then explanted after some period of time (e.g., a few months) after which the simulated exercise therapy is no longer needed, assuming the patient does not need other therapies delivered by the device.

A particularly suitable embodiment of a pacing device for delivering simulated exercise therapy on temporary basis is one that is incorporated entirely into a lead. That is, rather than having the pacing circuitry illustrated in FIG. 3 contained within a housing (or can, as it is usually called) implanted on a patient's chest, the pacing circuitry is contained within an intravascular lead. There are several advantages to integrating the entire pacing system into a lead. First, it leads to lower complication rates because there is no submuscular or subcutaneous pocket to become infected and/or irritated from the implanted housing. Second, most patients will more readily accept a pacing device without a bulky housing being implanted on their chest for both comfort and cosmetic reasons. Third, the lead/header connection is a common source of problems in conventional pacemakers, and a pacing device integrated into a lead has an inherent reliability advantage because there is such no header connection for the leads.

Integrating a pacing device into a lead requires that the components of the pacing device be small and able to be packaged into a relatively thin lead. In some embodiments, depending on the type of batteries used, the requirement that the batteries be small may limit the amount of energy that can be stored in them. In these embodiments, the pacing device will only function for some limited period of time and may be appropriately used only for temporary applications. As has been noted, one important temporary application of pacing therapy may be to deliver simulated exercise pacing. In other embodiments, the batteries are rechargeable, allowing the device to be used for permanent pacing. Recharging techniques such as acoustic and/or inductive coupling can be utilized. In still other embodiments, highly efficient batteries may be used to extend the time for which the integrated lead/pacing device may function so that other applications of pacing therapy with the device are appropriate. In any event, an integrated lead/pacing device as described below may be used not only for simulated exercise pacing but also for other applications such as conventional bradycardia pacing, cardiac resynchronization pacing, stress reducing post-MI pacing, or monitoring-only applications.

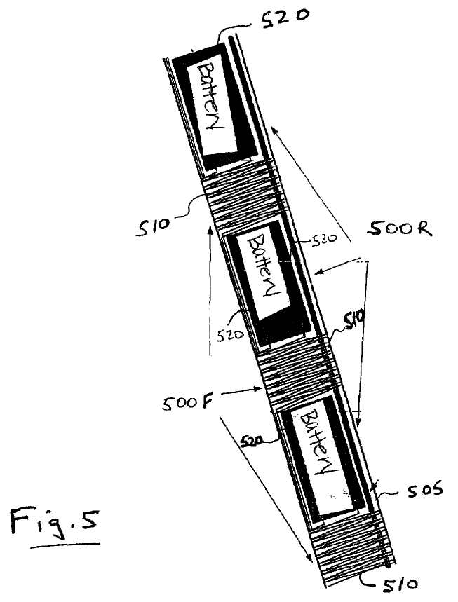

FIG. 5 shows in cross-section a middle portion of an integrated lead/pacemaker that is made up of a plurality of rigid lead sections 500R separated by a plurality of flexible lead sections 500F. The rigid sections 500R of the lead may contain circuitry components, while the flexible sections 500F may contain helical sections 510 of conductors that connect the circuitry components contained in the rigid sections. The flexible sections 500F enable the lead to be bent and navigated through a patient's vessel to a desired location. A lumen 505 for the implantation guide wire is provided within the lead which runs along the peripheral portion of the lead's interior to the side of the circuitry components within the lead. Shown in FIG. 5 as contained in the rigid sections 500R are a plurality of batteries 520 which may be connected in parallel. In one embodiment, the batteries are hermetically sealed ridged structures. In other embodiments the batteries are stacked thin film batteries, and in still other embodiments, the batteries are thin film, flexible batteries.

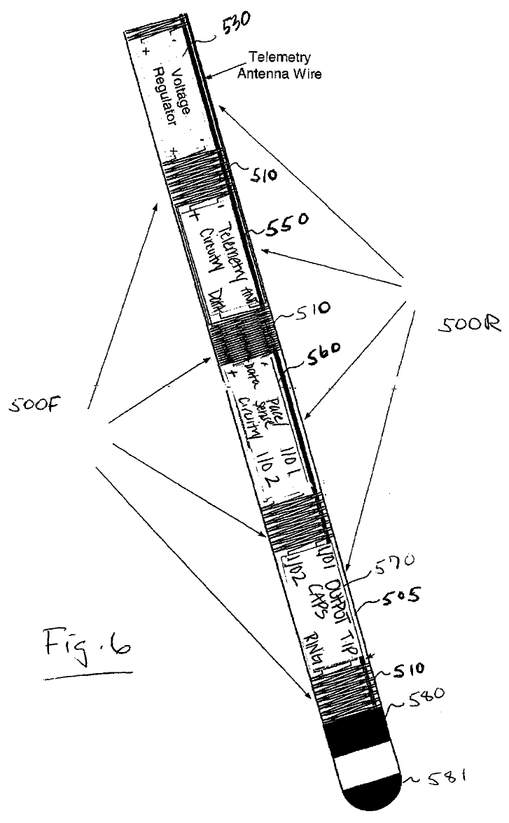

FIG. 6 shows a cross-section of the end portion of the integrated lead/pacemaker that includes a plurality of rigid sections 500R separated by a plurality of flexible sections 500F. The end portion of the lead contains the electronic modules that provide functionality to the device and that are contained in hermetically sealed compartments of the rigid sections 500R. The electronic modules shown in FIG. 6 include a power supply module 530 (which generates a regulated supply), a telemetry module 550, pace/sense circuitry modules 560, and an output pacing capacitor module 570. Other embodiments may include other electronic modules such as an accelerometer module (for rate adaptive pacing), and a battery-recharging module. Located at the distal end of the lead are a ring electrode 580 and a tip electrode 581. In one embodiment the pace/sense circuitry is located very close to the electrodes to minimize the amount of noise on the signal. This embodiment reduces the amount of filtering that needs to be done on the signal from that needed by traditional pacemakers.

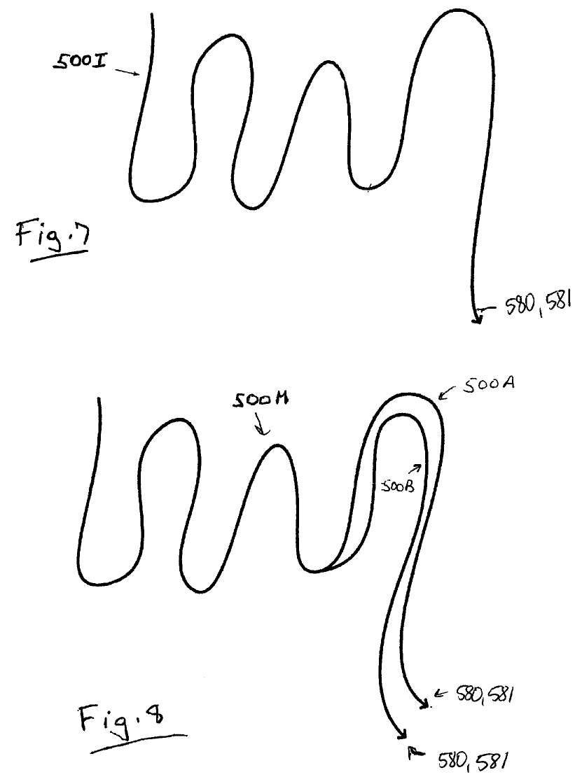

FIG. 7 shows a single chamber version of the integrated lead/pacemaker. In the embodiment shown, the proximal portion of the lead furthest from the electrodes is an inactive inert section 500I (made of silicone and/or polyurethane, possibly with a metal helical structure for strength). The inert section 500I is non-functional and allows the implanting physician to trim of excess lead length without impairing functionally. This allows the implanted lead to extend far enough that the end can be reached minimally invasively (subclavian approach) should it ever have to be removed, and yet not so far that the excess portion needs to be coiled in a sub-dermal pocket (as is common with traditional leads). In one embodiment the lead only delivers paces without sensing (e.g., VOO or AOO mode), while in another embodiment the lead both paces and senses (e.g., VVI or AAI mode). Both active and passive fixation versions of the pacing/sensing electrodes may be used. One lead may contain two electrodes; one electrode at the distal tip contacting the ventricle while a second electrode is located on the lead body more proximal about 4-10 cm from the distal tip to contact the atrium. This would allow the lead to both sense and stimulate both the atrium and ventricle.

FIG. 8 shows an embodiment that has a mid-portion 500M that bifurcates into two end portions 500A and 500B. Each of the two end portions 500A and 500B have pacing/sensing electrodes to enable multi-site pacing and/or sensing (e.g., dual-chamber pacing and/or sensing). A guide-wire can be steered selectively down a lumen in each of the two end portions 500A and 500B to allow each branch of the lead to be fixed to a desired location. Such a lead can support a DDD or DDDR pacing mode, for example. By branching into more than two lead end portions, more than two pacing and/or sensing sites can be achieved. Such a lead may be configured, for example, to deliver biventricular pacing. In another embodiment, a pacing system may be made up of two or more single-chamber pacing/sensing leads (such as shown in FIG. 7) that are separately implanted and coordinate their pacing by communicating wirelessly. Such a pacing system may be used for delivering dual-chamber pacing/sensing, biventricular pacing/sensing, or other multi-site pacing/sensing. The communication between the different leads of the pacing system can be done using acoustic telemetry techniques, E-field telemetry techniques, or RF telemetry techniques.

Although the invention has been described in conjunction with the foregoing specific embodiments, many alternatives, variations, and modifications will be apparent to those of ordinary skill in the art. Other such alternatives, variations, and modifications are intended to fall within the scope of the following appended claims.

What is claimed is:

1. A cardiac device, comprising:
one or more pacing channels for delivering pacing pulses to one or more myocardial sites;
a controller programmed to operate the device in either a normal operating mode or a simulated exercise mode;
wherein, in the simulated exercise mode, the controller is programmed to deliver paces to the one or more myocardial sites using a pacing mode that decreases cardiac output as compared with the normal operating mode; and,
wherein the controller is programmed to periodically or intermittently switch from the normal operating mode to the simulated exercise mode according to a defined schedule.

2. The device of claim 1 wherein the simulated exercise mode includes pacing a ventricular site using a VVI pacing mode with a ventricular escape interval shorter than a patient's intrinsic heart rate.

3. The device of claim 1 wherein the simulated exercise mode includes pacing a ventricular site using an atrial tracking or AV sequential pacing mode with an AV delay interval shorter than a patient's intrinsic AV delay interval.

4. The device of claim 1 wherein the simulated exercise mode includes pacing one or both ventricles at a site or sites that produce an asynchronous and inefficient ventricular contraction.

5. The device of claim 1 wherein the simulated exercise mode includes biventricular pacing with an interventricular pacing delay designed to produce inefficient cardiac contractions.

6. The device of claim 1 wherein the simulated exercise mode includes pacing an atrial or ventricular site at a rate that prevents adequate ventricular filling during diastole.

7. The device of claim 1 further comprising an exertion level sensor for measuring a patient's exertion level and wherein the controller is programmed to switch to the simulated exercise mode only if the measured exertion level is within a specified entry range.

8. The device of claim 1 further comprising a sensing channel for sensing cardiac activity and wherein the controller is programmed to switch to the simulated exercise mode only if a measured heart rate is within a specified entry range.

9. The device of claim 8 wherein the controller is programmed to switch to the simulated exercise mode only if no cardiac arrhythmia is detected.

10. The device of claim 1 further comprising a sensing channel for sensing cardiac activity and wherein the controller is programmed to detect cardiac ischemia and to switch to the simulated exercise mode only if no cardiac ischemia is detected.

11. The device of claim 1 further comprising an exertion level sensor for measuring a patient's exertion level and wherein the controller is programmed to switch from the simulated exercise mode to the normal operating mode if the measured exertion level is within a specified exit range.

12. The device of claim 1 further comprising a sensing channel for sensing cardiac activity and wherein the controller is programmed to switch from the simulated exercise mode to the normal operating mode if a measured heart rate is within a specified exit range.

13. The device of claim 1 further comprising a sensing channel for sensing cardiac activity and wherein the controller is programmed to switch from the simulated exercise mode to the normal operating mode if a cardiac arrhythmia is detected.

14. The device of claim 1 further comprising a sensing channel for sensing cardiac activity and wherein the controller is programmed to detect cardiac ischemia and to switch from the simulated exercise mode to the normal operating mode if cardiac ischemia is detected.

15. The device of claim 1 wherein the defined schedule specifies particular times of a day for switching to the simulated exercise mode.

16. The device of claim 1 wherein the defined schedule prescribes an amount of time over a specified time period for which the device is to operate in the simulated exercise mode and wherein the controller is programmed to opportunistically switch to the simulated exercise mode when one or more specified triggering conditions are met in order to meet the prescriptions of the defined schedule.

17. The device of claim 16 further comprising a patient actuated switch and wherein the one or more specified triggering conditions include the switch being actuated.

18. The device of claim 1 wherein the device includes pulse generation circuitry and sensing circuitry that incorporated along with the controller into a lead adapted for intravascular implantation.

19. A method for operating a cardiac pacing device, comprising:
delivering pacing pulses to one or more myocardial sites;
operating the device in either a normal operating mode or a simulated exercise mode;
in the simulated exercise mode, delivering paces to the one or more myocardial sites using a pacing mode that decreases cardiac output as compared with the normal operating mode; and,
periodically switching from the normal operating mode to the simulated exercise mode according to a defined schedule.

20. The method of claim 19 wherein the simulated exercise mode includes pacing a ventricular site using a VVI pacing mode with a ventricular escape interval shorter than a patient's intrinsic heart rate.

21. The method of claim 19 wherein the simulated exercise mode includes pacing a ventricular site using an atrial tracking or AV sequential pacing mode with an AV delay interval shorter than a patient's intrinsic AV delay interval.

22. The method of claim 19 wherein the simulated exercise mode includes pacing one or both ventricles at a site or sites that produce an asynchronous and inefficient ventricular contraction.

23. The method of claim 19 wherein the simulated exercise mode includes biventricular pacing with an interventricular pacing delay designed to produce inefficient cardiac contractions.

24. The method of claim 19 wherein the simulated exercise mode includes pacing an atrial or ventricular site at a rate that prevents adequate ventricular filling during diastole.
